(19) United States
(12) Patent Application Publication
(10) Pub. No.: US 2025/0078979 A1
(43) Pub. Date: Mar. 6, 2025

(54) DEVICE FOR PREDICTING AN OPTIMAL TREATMENT TYPE FOR THE PERCUTANEOUS ABLATION OF A LESION

(71) Applicant: Quantum Surgical, Montpellier (FR)

(72) Inventors: Lucien BLONDEL, Montpellier (FR); Bertin NAHUM, Castelnau-le-Lez (FR); Fernand BADANO, Lyon (FR)

(21) Appl. No.: 18/293,239

(22) PCT Filed: Oct. 3, 2022
(86) PCT No.: PCT/FR2022/051866

§ 371 Date: Jan. 29, 2024

(30) Foreign Application Priority Data
Oct. 7, 2021 (FR) FR2110648

(51) Int. Cl. G16H 20/40; A61B 18/00; G06T 7/00; G06T 7/10; G06T 7/62; G16H 10/60; G16H 30/40; G16H 50/70

(52) U.S. Cl. CPC G16H 20/40; A61B 18/00; G06T 7/0012; G06T 7/10; G06T 7/62; G16H 10/60; G16H 30/40; G16H 50/70; A61B 2018/00577; G06T 2207/10081; G06T 2207/10088; G06T 2207/10136; G06T 2207/30056; G06T 2207/30061; G06T 2207/30096; G06T 2207/30101

(57) ABSTRACT

The invention relates to a device for carrying out a method for selecting an optimal treatment for the percutaneous ablation of a lesion within an anatomical structure of interest of a patient. The method uses a machine learning algorithm trained to calculate, from a medical image on which the lesion can be seen, and for each of a plurality of available treatments, a confidence score, the value of which represents a likelihood of success of said treatment for the ablation of the lesion. The machine learning algorithm is trained beforehand using a set of training elements each comprising a medical image on which a lesion can be seen within the anatomical structure of interest of another patient, a treatment selected from the various available treatments for treating said other patient, and a confidence score for the selected treatment on said other patient. The optimal treatment is then selected on the basis of the confidence scores calculated for the different available treatments.

### FIELD OF THE INVENTION

The present patent application belongs to the field of the scheduling of a mini-invasive medical intervention. Notably, the application relates to an electronic device of computer type implementing a method for selecting an optimal treatment from among several available treatments for the percutaneous ablation of a lesion in an anatomy of interest of a patient.

### STATE-OF-THE-ART

To prepare a medical intervention aiming to treat a lesion (for example a tumor) in an anatomy of interest of a patient (for example a lung, a kidney, the liver, a bone structure, etc.), a practitioner generally performs a scheduling of the intervention based on a medical image.

The first step of this scheduling consists in selecting a treatment type. Some main clinical practice guidelines exist for selecting a treatment according to a type of pathology. The aim is, for example, initially to decide if it is best to resort to an ablation, a transplant, a treatment by chemoembolization, or a radiotherapy.

It does however remain difficult to appropriately select a more accurate treatment type.

More particularly, in the case of a percutaneous ablation of a tumor, various treatment methods can be applied: radiofrequency, microwave, laser, cryotherapy, electroporation or brachytherapy. The methods relying on radiofrequency waves, microwaves, laser or cryotherapy are thermal methods. Electroporation consists in delivering, between two electrodes, tens of radiofrequency pulses of a few μs at very high intensity to provoke irreversible alterations of the membrane functions of the cells. Electroporation is a relatively “gentle” ablation technology which makes it possible to consider treating tumors that could not be treated previously with the thermal ablation technologies, either because of the location of the tumor, or because of the fragility of the patient. Brachytherapy, for its part, aims to deliver radioactive doses inside or in proximity to the tumor.

Whatever the technique used (thermal energy, electrical energy, radioactive energy, etc.), a percutaneous ablation entails putting in place one or more applicators for delivering energy in situ. The energy delivered induces irreversible deconstructions on a cellular and tissue-level scale. In some cases, it may prove necessary to perform overlapped ablations sequentially by several successive insertions of an applicator, or simultaneously by the insertion and activation of several applicators at the same time.

Furthermore, when several applicators are activated simultaneously, there are various strategies for the deposition of energy by the applicators. A distinction is drawn notably between the so-called “centripetal convergent” ablations and the so-called “centrifugal” ablations.

The centrifugal ablation strategy is currently most widely used because it is relatively simple to implement. The centripetal convergent ablation strategy does however offer a better adaptability to the ablation zone and to its environment. The centripetal convergent ablation strategy indeed makes it possible to better control the destruction limits, which allows for a certain “modelling” of the ablation zones as a function of the form and the location of the tumors. On the other hand, it involves a more significant sacrifice of healthy tissues.

These various methods which, conceptually, may appear very similar, are in reality fairly different as much with regard to their practical use as to the results that they make it possible to obtain.

The choice of a particular treatment type is greatly dependent on the specific features of each clinical situation. Each particular treatment type can offer, in a particular clinical situation, the best benefit/risk ratio.

The choice of a particular treatment type is generally made by a practitioner according to his or her practices or preferences. Consequently, the treatment selected to treat a lesion in an anatomy of interest of a patient may not be optimal, and that may result in a recurrence of the pathology in the patient, or else collateral lesions.

Thus, there is however at the current time no satisfactory solution for assisting a practitioner in choosing the optimal treatment from among a set of available treatments for the ablation of a lesion in an anatomy of interest of a patient.

### SUMMARY OF THE INVENTION

The objective of the present invention is to wholly or partly remedy drawbacks of the prior art, notably those set out hereinabove.

To this end, and according to a first aspect, an electronic device is proposed comprising at least one processor and a computer memory. The computer memory stores program code instructions which, when they are executed by the processor, configure the processor to implement a method for selecting at least one optimal treatment from among several available treatments for the percutaneous ablation of a lesion within an anatomy of interest of a patient. The method implemented comprises:
obtaining of a medical image on which the lesion can be seen within the anatomy of interest of the patient,
calculation, for each of the different available ablation treatments, from the medical image and using a machine learning algorithm, of a confidence index whose value is representative of a probability of success of said treatment for the ablation of the lesion in the anatomy of interest of the patient, the machine learning algorithm having been trained beforehand from a set of training elements, each training element comprising:
   a medical image on which a lesion can be seen within the anatomy of interest of another patient,
   an identification of a treatment chosen from among the different ablation treatments available for treating said other patient,
   a confidence index of said treatment on said other patient,
selection of at least one optimal treatment as a function of the confidence indices calculated for the different available treatments.

“Percutaneous ablation” is understood to mean a mini-invasive intervention through the skin of the patient to treat a lesion in an anatomy of interest.

The anatomy of interest is for example the liver, a lung, a kidney, or a bone structure. The lesion is for example a tumor, a cyst or an aneurysm. Different treatments can be envisaged for ablating the lesion in the anatomy of interest. A treatment can notably be characterized by the choice of a technology (radiofrequency, microwave, laser, cryotherapy, electroporation, et cetera) and of an application method (for each technology, different energy deposition strategies can be envisaged).

The machine learning algorithm takes as input a medical image and a treatment type to provide directly as output a confidence index corresponding to a value representative of a probability of success of said treatment. The machine learning algorithm thus makes it possible to calculate, from the medical image of the anatomy of interest of the patient, a confidence index for each available treatment. An optimal treatment can then be selected as a function of the duly calculated confidence indices. For example, the treatment for which the confidence index is the highest corresponds to the treatment for which the probability of success of the treatment is the highest. The confidence index can notably be representative of a risk of recurrence. There may possibly be several possible optimal treatments, for example if different available treatments are associated with a confidence index of the same value.

The machine learning algorithm is trained beforehand based on a set of training elements. Each training element is for example associated with an intervention performed in the past on the anatomy of interest of another patient according to one of the available treatments. A training element then comprises a medical image of the anatomy of interest before treatment, an identification of the treatment which was performed on the patient, and a confidence index for this treatment (the confidence index is for example defined as a function of the observation or non-observation of a recurrence following the treatment).

A training element can however also be defined without an intervention having really taken place on another patient. In fact, the confidence index of a treatment envisaged to treat a lesion that can be seen on a medical image of the anatomy of interest of a patient can be determined theoretically. For example, the confidence index can be estimated by a panel of medical experts on the basis of the medical image. This estimation can possibly be weighted by recommendations from scientific publications and/or empirical studies conducted on groups of patients having similar lesions.

The set of training elements corresponds to a database that makes it possible to train the machine learning algorithm to calculate a confidence index for a particular treatment from a medical image showing a lesion to be treated. A medical establishment (hospital, clinic, et cetera) can create and enhance its own database for the treatments available within said medical establishment (for each new intervention performed in this establishment, a new training element can be added to the database).

In addition to the medical image, the machine learning algorithm can also be based on a set of parameters (metadata) relating to the lesion, to the anatomy of interest or to the patient. These parameters can be determined automatically on the medical image (for example via image segmentation or processing algorithms based on artificial intelligence), or else manually by a user. These parameters can correspond for example to the size of the lesion, to the position of the lesion with respect to the anatomy of interest or with respect to other anatomical structures, to a physical and medical condition of the patient, et cetera.

Different types of machine learning algorithms can be envisaged to implement the invention: decision tree forest (“random forest” in the literature), neural network, support vector machines (SVM), k-means partitioning, et cetera.

The device according to the invention offers various advantages. It notably makes it possible to obtain a reliable prediction of an optimal treatment for the percutaneous ablation of the lesion. The reliability of the prediction increases with the number of training elements. It is therefore preferable to train the machine learning algorithm from a large number of training elements. Also, it makes it possible to identify an optimal treatment very rapidly. In addition, the device according to the invention can be used by people who are not necessarily experts in percutaneous ablation (for example by interns or doctors being trained for this practice). The proposed solution therefore makes it possible to democratize treatments by percutaneous ablation.

In particular implementations, the device can further comprise one or more of the following features, taken alone or in all technically possible combinations.

In particular implementations, each available treatment is characterized by an ablation technology chosen from among radiofrequencies, microwaves, laser, cryotherapy, electroporation or brachytherapy.

In particular implementations, each available treatment is characterized by a strategy of energy deposition by one or more applicators, said strategy being chosen from among an energy deposition of centrifugal type and an energy deposition of centripetal convergent type.

In particular implementations, each available treatment is characterized by a number of applicators, and/or by the positions of said applicators with respect to the tumor.

In particular implementations, for each training element, the confidence index is defined as a function:
of the observation or non-observation of a recurrence for said other patient with the chosen treatment, and/or
of a duration of the period elapsed between the end of the treatment and a current date without a recurrence having been observed for said other patient, and/or
of a duration of the period elapsed between the end of the treatment and a recurrence for said other patient, and/or
of an estimation, by one or more medical experts, of a probability of success of the chosen treatment on said other patient.

In particular implementations, the method comprises an association of a set of parameters with the medical image of the patient, said set of parameters comprising one or more parameters relating to the lesion, to the anatomy of interest and/or to characteristics of the patient. Each training element comprises a set of similar parameters relating to the lesion, to the anatomy of interest and/or to characteristics of the other patient for which the medical image corresponding to said reference element was obtained.

In particular implementations, the set of parameters comprises one or more parameters chosen from among:
the age, the sex, the weight and/or the height of the patient for whom the medical image was obtained,
a comorbidity presented by the patient for whom the medical image was obtained,
a value representative of the size and/or of a volume of the lesion,
a distance between the lesion and a capsule of the anatomy of interest,
a distance between the lesion and a blood vessel close to the lesion,
when the anatomy of interest is the liver, a distance between the lesion and a hepatic duct close to the lesion.

In particular implementations, the method comprises a transformation of the medical image of the patient, the medical image of each training element having undergone a similar transformation before being used to train the machine learning algorithm.

In particular implementations, the transformation of the medical image comprises:
a segmentation of the lesion on the medical image, and/or
a segmentation of the anatomy of interest on the medical image, and/or
a segmentation of blood vessels on the medical image, and/or
when the anatomy of interest is the liver, a segmentation of hepatic ducts on the medical image.

In particular implementations, the transformation of the medical image comprises a reframing of the image around the lesion according to a frame whose dimensions are predetermined, said frame being common to all the medical images of the training elements, the position of the lesion with respect to the frame being variable from one medical image to the other.

In particular implementations, the method comprises:
for each training element corresponding to the selected optimal treatment, a calculation of a similarity value representative of the similarity between the medical image of the patient to be treated and the medical image of the training element, and
a selection of a reference training element from among the set of the training elements as a function of the calculated similarity values.

In particular implementations, the medical images were acquired by tomodensitometry, by magnetic resonance imaging or by ultrasound.

In particular implementations, the medical images are three-dimensional.

In particular implementations, the anatomy of interest is the liver, a kidney or a lung, and the lesion is a tumor or a cyst.

In particular implementations, the set of training elements comprises training elements whose medical images do not show a lesion in the anatomy of interest.

### DESCRIPTION OF THE FIGURES

The invention will be better understood on reading the following description, given as a nonlimiting example, and by referring to FIGS. 1 to 4 which represent:

FIG. 1 a schematic representation of the main steps of a method for selecting an optimal treatment for the ablation of a lesion in an anatomy of interest of a patient.

FIG. 2 a schematic representation of an electronic device allowing the selection of an optimal treatment for the ablation of a lesion in an anatomy of interest of a patient,

FIG. 3 a schematic representation of a step of collection of a set of reference elements used to train the machine learning algorithm,

FIG. 4 a schematic representation of a particular implementation of a method for selecting an optimal treatment, in which a reference training element is selected.

In these figures, references that are identical from one figure to another designate identical or analogous elements. For reasons of clarity, the elements represented are not necessarily to a same scale, unless stipulated otherwise.

### DETAILED DESCRIPTION OF AN EMBODIMENT OF THE INVENTION

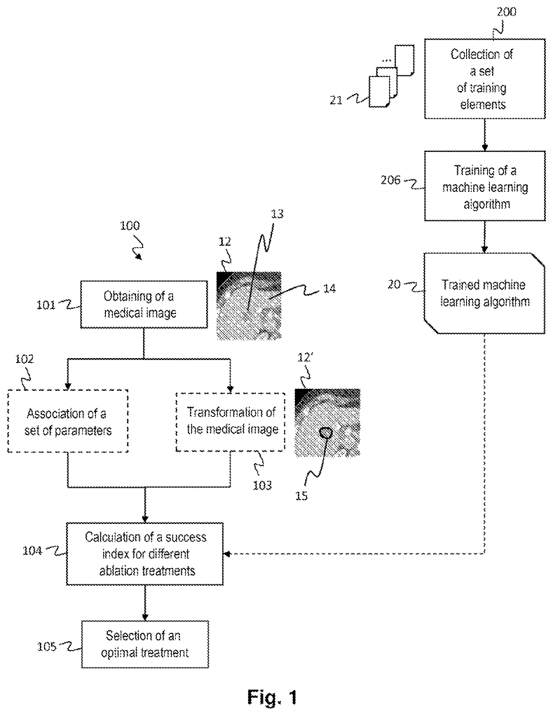

FIG. 1 schematically represents the main steps of a method 100 for selecting an optimal treatment for the ablation of a lesion in an anatomy of interest of a patient. The optimal treatment is chosen from among several available treatments. It should be noted that several treatments can possibly be indicated as optimal by the method 100 without it being possible to decide between them.

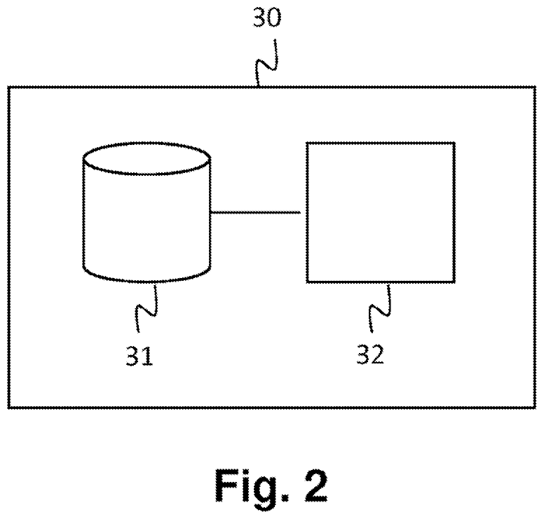

The method 100 is implemented by an electronic device of computer, tablet, cellphone and other such type. FIG. 2 schematically illustrates an exemplary embodiment of such an electronic device 30. The electronic device 30 comprises at least one processor 32 and a computer memory 31 storing program code instructions which, when they are executed by the processor 32, configure the processor 32 to implement the method 100 for selecting a treatment according to the invention.

The computer memory 31 therefore corresponds to a storage medium that can be read by the electronic device 30, and comprising instructions which, when they are executed by said electronic device 30, cause the latter to implement the method 100 for selecting a treatment.

The invention can also take the form of a computer program comprising instructions which, when the program is run by the electronic device 30, cause the latter to implement the method 100 for selecting a treatment.

The method 100 comprises a step of obtaining 101 of a medical image 12 on which the lesion 13 can be seen within the anatomy of interest 14 of the patient.

In the example considered, the medical image 12 is a two-dimensional medical image acquired by tomodensitometry (“CT-scan” for “Computerized Tomography Scan” in the literature). There is however nothing to prevent the medical image from being acquired according to another medical imaging modality, for example by MRI (“Magnetic Resonance Imaging”) or by ultrasound. Nor is there anything to prevent the medical image from being a three-dimensional image.

The medical image 12 is for example stored in the computer memory 31 of the electronic device 30 and processed by the processor 32. The medical image 12 is for example transmitted to the electronic device 30 via wired communication means or wireless communication means (these means are not represented in FIG. 2). According to another example, the electronic device 30 can be connected to an external memory peripheral device, for example a USB (acronym for “Universal Serial Bus”) stick on which is stored the medical image 12. In any case, the electronic device 30 is configured to obtain the medical image 12 which was previously acquired on the patient to be treated.

In the example considered, and in a nonlimiting manner, the anatomy of interest is the liver, and the lesion is a cancerous tumor. The invention could however also be applied to other anatomies of interest (for example a lung, a kidney, a bone structure, et cetera), and/or to other types of lesions (for example a cyst, an aneurysm, a metastasis, et cetera).

The available treatments, among which at least one optimal treatment is selected, correspond for example to different treatments used in a medical establishment to treat such a tumor.

An available treatment can notably be characterized by an ablation technology chosen from among radiofrequency, microwave, laser, cryotherapy, electroporation or brachytherapy. These various technologies are known to the person skilled in the art for treating a cancerous tumor in the liver.

An available treatment can further be characterized by a strategy of energy deposition by one or more applicators (needle, electrode, probe, et cetera). The energy deposition strategy can notably be chosen from among an energy deposition of centrifugal type and an energy deposition of centripetal convergent.

For a long time, the preferred energy deposition strategy followed a centrifugal coverage scheme in which the energy is delivered isotropically from the center of the tumor (where the applicator is inserted) to the periphery of the tumor. In some cases, it may prove necessary to perform overlapped ablations sequentially by several successive activations of an applicator inserted at different positions, or simultaneously by activation at the same time of several applicators at different positions. The deposition strategy of centrifugal type offers the advantage of being a technique that is particularly simple and proven. However, in some particular conditions (as a function notably of the size or of the volume of the tumor, or as a function of the tissue properties of the lesion or of the anatomical structures close to the lesion), the strategy of centrifugal type is not always optimal. Another energy deposition strategy, consisting in making the energy converge from the periphery to the center of the tumor (strategy of centripetal convergent type) is then sometimes preferable.

In addition to the ablation technology and the energy deposition strategy, an available treatment can further be characterized by the number of applicators used, and/or by the positions of said applicators with respect to the tumor.

The position of the applicators is generally determined with respect to the tumor, but it can also be determined with respect to the environment of the tumor (that is to say the anatomical structures close to the tumor: bone, blood vessels, organs at risk, et cetera).

The method 100 comprises a step of calculation 104, for each of the different available ablation treatments, of a confidence index whose value is representative of a probability of success of said treatment for the ablation of the lesion 13 in the anatomy of interest 14 of the patient. The confidence indices of the different available treatments are calculated from the medical image 12 by a machine learning algorithm 20 trained beforehand.

As illustrated in FIG. 1, the machine learning algorithm is trained, during a training step 206, from a set of training elements 21. Each training element 21 is for example associated with an intervention performed in the past to treat a lesion in the anatomy of interest of another patient according to one of the available treatments. A training element 21 then comprises a medical image of the anatomy of interest of said other patient before treatment, an identification of the treatment which was performed on said other patient, and a confidence index for this treatment.

For each training element 21, the confidence index can be defined for example as a function of the observation or non-observation of a recurrence for said other patient with the chosen treatment (if there has not been any recurrence, the probability of success of the treatment is relatively strong: conversely, if there has been a recurrence, the probability of success of the treatment is lower).

The duration of the period elapsed between the end of the treatment and the current date without a recurrence having been observed can also be a factor used to define the confidence index of the treatment (the longer this period is, the greater the probability of success of the treatment).

If there has been a recurrence, it is possible to take account of the duration of the period elapsed between the end of the treatment and the recurrence to determine the confidence index (the shorter this period is, the lower the confidence index).

It should be noted that the step of collection 200 of the training elements and the step of training 206 of the machine learning algorithm are performed prior to the method 100 for selecting an optimal treatment (they do not therefore form part of the method 100 for selecting an optimal treatment).

It should also be noted that the training elements 21 can also be obtained without interventions having really taken place on other patients. Indeed, the confidence index of a treatment envisaged for treating a lesion that can be seen on a medical image of the anatomy of interest of another patient can be determined theoretically by a panel of medical experts.

Once the confidence indices have been calculated by the machine learning algorithm for the different available treatments, it becomes possible to select, during a selection step 105, at least one optimal treatment out of the different available treatments. For example, the optimal treatment corresponds to the treatment for which the calculated confidence index is the highest. As indicated previously, there can possibly be several possible optimal treatments, for example if different available treatments are associated with a confidence index of the same value.

The set of training elements 21 corresponds to a database used to train the machine learning algorithm to calculate a confidence index for a particular treatment from a medical image showing a lesion to be treated. This database can be enriched over time, each time a new medical image showing a lesion in the anatomy of interest of a patient is available and a confidence index has been determined for a particular treatment of this lesion.

It is possible to envisage applying a selection on the cases likely to be used to form training elements 21. In particular, when the training elements 21 are constructed on the basis of interventions having actually taken place, it is advantageous to consider only the interventions for which the ablation margin was sufficient to entirely cover the tumor. Indeed, for an intervention in which the ablation margin would not have been sufficient, a recurrence of the tumor could be attributed to the chosen treatment type, whereas the recurrence could primarily be due to an insufficient ablation margin. The ablation margin is for example defined as the smallest distance between the periphery of the lesion and the periphery of the ablation region. It is generally appropriate for the ablation region to entirely cover the lesion, and for the ablation margin to be at least equal to a threshold value, for example five millimeters.

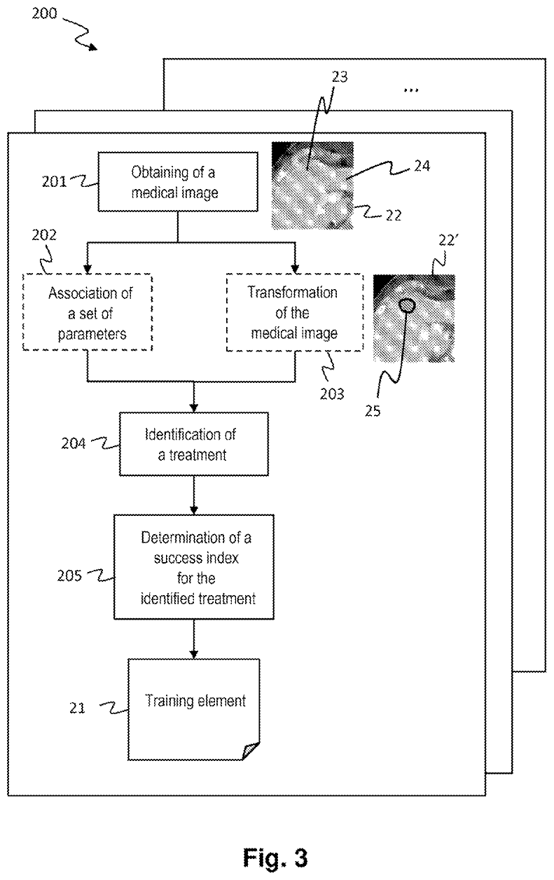

FIG. 3 schematically represents the collection 200 of the training elements used to train the machine learning algorithm.

To obtain a training element 21, the first step should be to collect (step 201) a medical image 22 on which a lesion 23 can be seen within the anatomy of interest 24 of another patient.

It can be for example another patient on which an intervention has taken place in the past to ablate the lesion 23 with a particular treatment from among the different available treatments. Preferably, this intervention has taken place within the same medical establishment, but it could also have taken place in another establishment. However, the treatment applied for the intervention should correspond to one of the available treatments. As indicated previously, it is not however essential for an intervention aiming to treat the lesion to have really taken place.

Next, a particular treatment should be identified (step 204), from among the different available treatments, to treat the lesion 23. If an intervention has taken place, it is the treatment which was chosen to ablate the lesion 23 (this choice was able to be made, notably, using the selection method according to the invention, or else from a study of the medical image 22 by one or more medical experts). It can also be treatment which is estimated as being the most appropriate by one or more medical experts based on the medical image 22.

Finally, a confidence index should be determined (step 205) for the chosen treatment. This confidence index can be determined according to different methods described previously. If an intervention has taken place, the confidence index can notably be defined as a function of the observation or non-observation of a recurrence following the treatment, as a function of the duration of the period elapsed between the end of the treatment and the current date without a recurrence having been observed, or as a function of the duration of the period elapsed between the end of the treatment and the recurrence, et cetera. The confidence index can also be estimated by one or more medical experts on the basis of the medical image (notably in the case where there has not yet been any intervention on the lesion 23, or if it is not possible to obtain information on the occurrence or non-occurrence of a recurrence after the intervention).

The machine learning algorithm 20 can also be based on a set of parameters (metadata) relating to the lesion, to the anatomy of interest or to the patient. That makes it possible to reinforce the accuracy of the prediction made by the machine learning algorithm 20.

The parameters are for example determined manually by a user. Alternatively, or in addition, the parameters can be determined automatically on the medical image (for example via image segmentation or processing algorithms based on artificial intelligence).

These parameters can notably correspond to physical or medical characteristics of the patient: the age, the sex, the weight and/or the height of the patient, or the identification of certain comorbidities suffered by the patient (heart failure, immunodeficiency, alcoholism, et cetera). They can also correspond to a value representative of the size or of the volume of the lesion, to a distance between the lesion and a capsule of the anatomy of interest, or to a distance between the lesion and certain anatomical structures close to the lesion (blood vessel, hepatic duct, et cetera). All these characteristics can in fact have an impact on the effectiveness or the risks associated with a particular treatment (certain treatments are not indicated for lesions of large sizes, the presence of blood vessels in proximity to the lesion impacts the effectiveness of the thermal treatments, some treatments are too risky for fragile patients, et cetera).

Preferably, the same parameters used to create the training elements 21 are also used during the method 100 for selecting an optimal treatment. To this end, and as illustrated in FIGS. 1 and 3, the step of collection 200 of training elements 21 comprises, for each training element 21, a step of association 202 of a set of parameters with the medical image 22 corresponding to said training element 21. The method 100 also comprises a step of association 102 of a set of parameters similar to the medical image 12 of the patient to be treated. The sets of parameters associated respectively with the medical images 22 of the training elements 21, and the set of parameters associated with the medical image 12 of the patient to be treated are all similar to one another. This means that the sets of parameters all comprise parameters of the same type (however, the values of the parameters are generally different from one set of parameters to the other).

As illustrated in FIG. 3, the collection of a training element 21 can also comprise a step of transformation 203 of the medical image 22 corresponding to said training element 21. This transformation 203 can correspond for example to a segmentation 25 of the lesion 23 on the medical image 22. According to other examples, the transformation 203 could also correspond to a segmentation of the anatomy of interest 24 and/or of particular anatomical structures on the medical image 22 (blood vessels, hepatic ducts, et cetera). In the example illustrated in FIG. 3, the transformed image 22′ obtained comprises a segmentation 25 of the lesion. As illustrated in FIG. 1, the method 100 for selecting an optimal treatment comprises a similar step of transformation 103 of the medical image 12 of the patient to be treated. In the example illustrated in FIG. 1, the transformed image 12′ obtained comprises a segmentation 15 of the lesion 13.

In particular implementations, the transformation 103, 203 of a medical image 12, 22 comprises a reframing of the image 12, 22 around the lesion 13, 23 according to a frame whose dimensions are predetermined, for example 128×128 pixels. The dimensions of the frame are the same for all the medical images 22 of the training elements 21. However, and advantageously, the position of the lesion 23 with respect to the frame varies from one medical image 22 to the other. Such provisions make it possible to reduce the prediction errors of the machine learning algorithm 20 (it is indeed essential to avoid having the algorithm learn that the lesion to be treated is mainly at the center of the medical image, which is not necessarily always true).

The step of association 102, 202 of a set of parameters and the step of transformation 103, 203 of the medical image 12, 22 are optional (that is why they are represented by dotted lines in FIGS. 1 and 3). The machine learning algorithm 20 can in fact be based exclusively on the information contained in the medical images 12, 22. The step of association 102, 202 of a set of parameters and/or the step of transformation 103, 203 of the medical image 12, 22 do however make it possible to give additional information to the machine learning algorithm 20, thus enhancing the accuracy of the prediction.

Different types of machine learning algorithms can be envisaged for implementing the selection method 100 according to the invention. The machine learning algorithm must predict a confidence index for each treatment available for ablating the lesion 13 that can be seen in the medical image 12 acquired on the patient to be treated.

Let Ω={(Xᵢ, Tᵢ, Iᵢ)|i=1:N) be a set of training elements 21, in which:
Xᵢ=[xᵢ₁, xᵢ₂, . . . , xᵢⱼ, . . . , x_{iK}] is a point of a space with K dimensions corresponding to K characteristics extracted from the medical image 22 associated with the training element of index i (these parameters can possibly also be extracted from the transformed medical image 22′ and/or from the set of parameters associated with the training element of index i),
Tᵢ is a particular treatment from among the different available treatments,
Iᵢ is the confidence index representative of a probability of success of the treatment Tᵢ for the ablation of the lesion that can be seen on the medical image 22 associated with the training element of index i,
N is the number of elements in the set of training elements considered.

The confidence index Iᵢ corresponds for example to a score out of ten (for example, the index Iᵢ takes an integer value lying between 0 and 10). According to another example, the confidence index Iᵢ can correspond to a normalized value between 0 and 1 representative of a probability of success of the treatment Tᵢ.

If the value of N is sufficiently great (for example at least equal to a thousand, even at least equal to five thousand), it is possible to train a machine learning algorithm to calculate a value I, for each available treatment T, from a set X of characteristics linked to a medical image of a patient to be treated (and possibly to the patient him or herself).

To this end, machine learning algorithms of “neural network” type can be used. At least two different methods can be envisaged. A first method consists in applying the different neurons to the different points Xi in order to predict a value Iᵢ for a particular treatment Tᵢ. In this case, the characteristics xᵢⱼ are defined by hand (or “hand-crafted”). That does however necessitate a deep knowledge of the domain. Another method consists in delegating the choice of the characteristics to the neural network. In this second method, the input of the neural network is the medical image 12 on which the lesion 13 to be treated can be seen, and the characteristics are extracted by the convolution filters of the neural network defined during the training process. The use of additional characteristics (metadata) not forming part of the medical image 12 is however still possible. These additional characteristics can notably be introduced at the last layer of the neural network.

Machine learning algorithms of “random forest” type can also be used. The classification trees form part of the algorithms that are the most popular in the field of machine learning and they are the basis of the most powerful methods. For example, in a tree structure, a node selects the variable xij which minimizes a classification error according to a certain rule, and a leaf represents a confidence index Iᵢ for a particular treatment Tᵢ. The set of training elements is used to define the variable xij and the specific rule to be used for each node. For example, the first node of the tree could correspond to the volume of the lesion and to the “volume of the tumor less than 4 cm³” rule. If the volume is less than 4 cm³, a second node could then for example correspond to a choice on the type of the lesion (hepatocellular carcinoma, metastasis, et cetera). The path through the nodes of the tree (corresponding to the verification of a set of rules) makes it possible to culminate in a prediction value for the confidence index Iᵢ for a treatment Tᵢ. Multiple classification trees can be created for the different treatments available by a random sampling process based on the set Ω of training elements 21. For a new value of X, each tree gives a value of I and the final prediction can then be made by a simple majority vote. Each tree is generally constructed independently of the others and has the same weight on the final decision.

According to another example, a machine learning algorithm of “AdaBoost” type can be used (abbreviation of the term “Adaptive Boosting”). The conventional implementation of the AdaBoost method consists in a combination of low classifiers (typically of the classification trees with a single node) in which subsequent low classifiers are adjusted to give more weight to the samples badly classified by the preceding classifiers. Unlike the random forests, in the AdaBoost method the low classifiers are not independent of the others and nor do they have the same weight on the final classification. In the issue addressed here, a first low classifier would be composed for example of a single node corresponding to the volume of the lesion, and the next classifier, which would for example have a single node corresponding to the type of lesion, would be created to compensate the classification errors of the first classifier.

According to another example, a machine learning algorithm of “support vector machine” type can be used. This technique for example makes it possible to separate, in a binary manner, the points X corresponding to patients treated by a particular treatment with a first confidence index I (corresponding for example to a probability of success greater than a certain threshold), from the points X′ corresponding to patients treated with the same treatment but with a different confidence index I′ (corresponding for example to a probability of success less than said threshold). For that, kernel functions are applied to the different points in order to render them separable by a hyper-plane in a space of greater dimension. This technique can thus make it possible to predict a confidence index (informing on the probability of success with respect to a predetermined threshold) for a particular treatment (the support vector machines are binary classifiers). In order to be able to manage a finer granularity of the confidence index and several possible treatments, the problem can be subdivided into several binary problems (approach referred to as “One-vs-One and One-vs-Rest”).

According to yet another example, a machine learning algorithm of “k-means” type can be used. This is an unsupervised learning method which groups together the training data Xᵢ in different groups (“clusters”) represented by their averages [symbol] , (p varying for example from 1 to M, in which M is the number of groups) which minimizes the sum S of the distances between the points X of a group and the associated average [symbol] :

$$S = {\sum }_{p = 1}^{M} {\sum }_{X ∈ p} ‖ X - \hat{{X}_{p}} ‖ \qquad [ Math . 1 ]$$

In the case concerned here, for a given treatment, M corresponds for example to a number of possible values taken by the confidence index (for example M=11 if the confidence index corresponds to an integer value varying between 0 and 10). Once the algorithm is trained, the confidence index for a new patient represented by X is determined by seeking the average [symbol] closest to X.

Other types of machine learning algorithms could be envisaged, both classification algorithms and regression algorithms. The choice of a particular type of machine learning algorithm is only a variant of the invention.

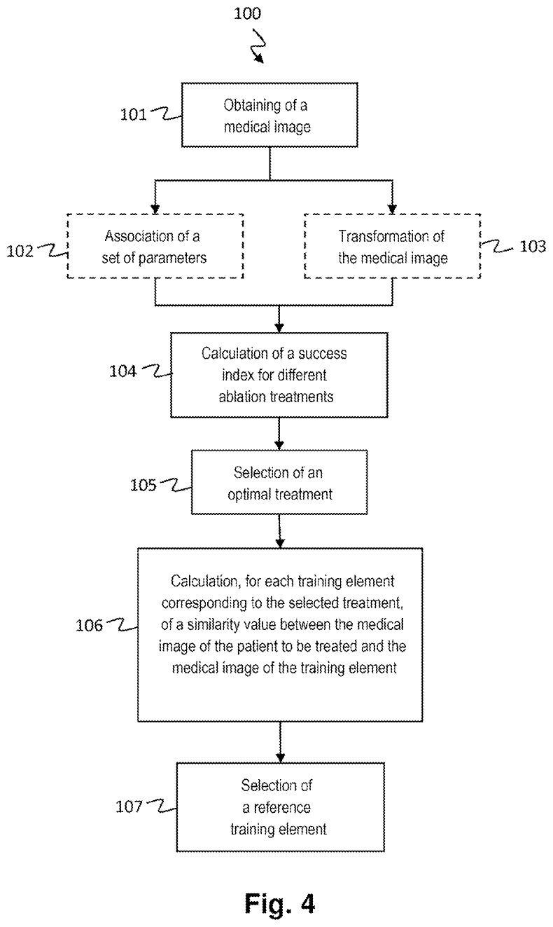

It may be advantageous, for the practitioner who will perform the treatment, to obtain a reference medical image corresponding to a particularly similar case having been previously treated with the same treatment as the selected optimal treatment. To this end, in a particular implementation as illustrated in FIG. 4, the method 100 comprises a step of calculation 106 of a similarity value for each training element 21 corresponding to the selected optimal treatment. The similarity value is representative of the similarity between the medical image 12 of the patient to be treated and the medical image 22 of the training element 21 considered. The method 100 then comprises a step of selection 107 of a reference training element from among the set of the training elements 21 as a function of the calculated similarity values. For example, the reference element corresponds to the training element 21 having the greatest similarity value. The medical image 22 associated with this reference element can then be used by the practitioner to compare the clinical case of the patient to be treated with the clinical case corresponding to the reference element. A high similarity value indicates that the lesion that can be seen on the medical image of the reference element is similar to the lesion that can be seen on the medical image of the patient to be treated. That can make it possible to reassure the practitioner as to the validity of the selected optimal treatment. The practitioner can also take information on the progress or the results of a possible intervention having taken place to treat the lesion of the case corresponding to the reference element.

It may be that the benefit/risk ratio of conducting an intervention on the patient to ablate the lesion is not sufficient (if the beneficial therapeutic effects of the ablation are lower than the risks linked to the implementation thereof). In such a case, it is preferable not to ablate the lesion. It is therefore possible to envisage that the option consisting in not ablating the lesion forms part of the available treatments. In other words, the optimal treatment selected by the method 100 can consist in not treating the lesion. That would also be the case for example if the element identified as a lesion by the practitioner on the medical image 12 of the patient is in fact not a lesion, or if it is a benign lesion.

For these reasons, it may be advantageous to train the machine learning algorithm 20 with medical images 22 on which there is no lesion to be seen in the anatomy of interest. Thus, in particular implementations, the medical images 22 associated with some of the training elements 21 used to train the machine learning algorithm 20 do not show any lesion that can be seen in the anatomy of interest.

The above description clearly illustrates that, through its various features and the advantages thereof, the present invention achieves the objectives set. In particular, the invention makes it possible to obtain a reliable and rapid prediction of an optimal treatment for the percutaneous ablation of a lesion in the anatomy of interest of a patient. Furthermore, the invention can be used by people who are not necessarily experts in percutaneous ablation; the proposed solution therefore allows for a democratization of the treatments by percutaneous ablation.

1. An electronic device comprising at least one processor and a computer memory storing program code instructions which, when they are executed by said at least one processor, configure said at least one processor to implement a method for selecting at least one optimal treatment from among several available treatments for the percutaneous ablation of a lesion within an anatomy of interest of a patient, said method comprising:
obtaining a medical image previously acquired on the patient and on which the lesion can be seen within the anatomy of interest of the patient,
calculating using a machine learning algorithm, from the medical image, for each of the different available ablation treatments, a confidence index whose value is representative of a probability of success of said treatment for the ablation of the lesion in the anatomy of interest of the patient, the machine learning algorithm having been trained beforehand from a set of training elements, each training element comprising:
a medical image on which a lesion can be seen within the anatomy of interest of another patient,
an identification of a treatment selected from among the different ablation treatments available for treating said other patient, and
a confidence index of said treatment on said other patient, and
selecting at least one optimal treatment as a function of the confidence indices calculated for the different available treatments.

2. The electronic device of claim 1, wherein each available treatment is an ablation technology selected from radiofrequencies, microwaves, laser, cryotherapy, electroporation or brachytherapy.

3. The electronic device of claim 2, wherein each available treatment uses a strategy of energy deposition by one or more applicators, said strategy selected from an energy deposition of centrifugal type or an energy deposition of centripetal convergent type.

4. The electronic device of claim 3, wherein each available treatment is characterized by a number of applicators, and/or by the positions of said applicators with respect to the lesion.

5. The electronic device of claim 1, wherein, for each training element the confidence index is defined as a function:
of the observation or non-observation of a recurrence for said other patient with the chosen treatment, and/or
of a duration of the period elapsed between the end of the treatment and a current date without a recurrence having been observed for said other patient, and/or
of a duration of the period elapsed between the end of the treatment and a recurrence for said other patient, and/or
of an estimation, by one or more medical experts, of a probability of success of the chosen treatment on said other patient.

6. The electronic device of claim 1, wherein the method comprises associating a set of parameters with the medical image of the patient, said set of parameters comprising one or more parameters relating to the lesion, to the anatomy of interest and/or to characteristics of the patient, each training element comprising a set of similar parameters relating to the lesion, to the anatomy of interest and/or to characteristics of the other patient for which the medical image corresponding to said reference element was obtained.

7. The electronic device of claim 6, wherein the set of parameters comprises one or more parameters selected from:
the age, the sex, the weight and/or the height of the patient for whom the medical image was obtained,
a comorbidity presented by the patient for whom the medical image was obtained,
a value representative of the size and/or of a volume of the lesion,
a distance between the lesion and a capsule of the anatomy of interest,
a distance between the lesion and a blood vessel close to the lesion, and or
when the anatomy of interest is the liver, a distance between the lesion and a hepatic duct close to the lesion.

8. The electronic device of claim 1, wherein the method comprises transforming the medical image of the patient, the medical image of each training element having undergone a similar transformation before being used to train the machine learning algorithm.

9. The electronic device of claim 8, wherein transforming the medical image comprises:
segmenting the lesion on the medical image, and/or
segmenting the anatomy of interest on the medical image, and/or
segmenting blood vessels on the medical image, and/or
when the anatomy of interest is the liver, segmenting hepatic ducts on the medical image.

10. The electronic device of claim 8, wherein transforming the medical image comprises reframing of the image around the lesion according to a frame whose dimensions are predetermined, said frame being common to all the medical images of the training elements, the position of the lesion with respect to the frame being variable from one medical image to the other.

11. The electronic device of claim 1, wherein the method comprises:
for each training element corresponding to the selected optimal treatment, calculating a similarity value representative of the similarity between the medical image of the patient to be treated and the medical image of the training element, and
selecting a reference training element from among the set of the training elements as a function of the calculated similarity values.

12. The electronic device of claim 1, wherein the medical images were acquired by tomodensitometry, by magnetic resonance imaging or by ultrasound.

13. The electronic device of claim 1, wherein the medical images are three-dimensional.

14. The electronic device of claim 1, wherein the anatomy of interest is the liver, a kidney or a lung, and the lesion is a tumor or a cyst.

15. The electronic device of claim 1, wherein the machine learning algorithm is further trained beforehand with training elements whose medical images do not show any lesion in the anatomy of interest.
